(19) United States
(12) Patent Application Publication
(10) Pub. No.: US 2014/0221389 A1
(43) Pub. Date: Aug. 7, 2014

(54) USE OF 4-AMINO-5-FLUORO-3-[6-(4-METHYLPIPERAZIN-1-YL)-1H-BENZIMIDAZOL-2-YL]-1H-QUINOLIN-2-ONE IN THE TREATMENT OF CANCER IN MODERATE HEPATIC IMPAIRED PATIENTS

(71) Applicants: Suraj Anand, Parsippany, NJ (US); Catherine Reddick, Orange, NJ (US); Michael Shi, Chalfont, NJ (US); Mary Steed, Short Hills, NJ (US); Mario Stegert, Riehen (CH); Eugene Tan, Basking Ridge, NJ (US); Yongyu Wang, Morristown, NJ (US)

(72) Inventors: Suraj Anand, Parsippany, NJ (US); Catherine Reddick, Orange, NJ (US); Michael Shi, Chalfont, NJ (US); Mary Steed, Short Hills, NJ (US); Mario Stegert, Riehen (CH); Eugene Tan, Basking Ridge, NJ (US); Yongyu Wang, Morristown, NJ (US)

(73) Assignee: Novartis AG, Basel (CH)

(21) Appl. No.: 14/342,612

(22) PCT Filed: Sep. 7, 2012
(86) PCT No.: PCT/US2012/054046

§ 371 Date: Mar. 4, 2014

Related U.S. Application Data
(60) Provisional application No. 61/535,142, filed on Sep. 15, 2011.

(51) Int. Cl. A61K 31/496; A61K 31/19

(52) U.S. Cl. CPC A61K 31/496; A61K 31/19
USPC 514/253.07

(57) ABSTRACT

The present invention relates to a method of treating a cancer in patient in need thereof by administering 4-amino-5 fluoro-3-[6-(4-methylpiperazin-1-yl)-1H-benzimidazol-2-yl]-1H-quinolin-2-one, or a tautomer thereof, or a salt of any one of them, wherein the patient is a moderate hepatic impaired patient.

The present invention relates to a method of treating a cancer in a patient in need thereof by administering 4-amino-5-fluoro-3-[6-(4-methylpiperazin-1-yl)-1H-benzimidazol-2-yl]-1H-quinolin-2-one, or a tautomer thereof, or a salt of any one of them (COMPOUND I), wherein the patient is a moderate hepatic impaired patient.

The following description is provided to assist the understanding of the reader. None of the information provided or references cited herein are admitted to be prior art to the present technology.

4-Amino-5-fluoro-3-[6-(4-methylpiperazin-1-yl)-1H-benzimidazol-2-yl]-1H-quinolin-2-one has the structure shown in Formula I:

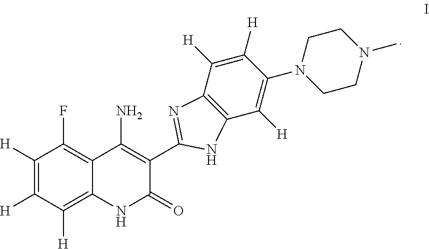 [SMILES: CN1CCN(c2ccc3nc(-c4c(N)c5c(F)cccc5[nH]c4=O)[nH]c3c2)CC1]

The compound of Formula I inhibits various protein kinases, such as tyrosine receptor protein kinases. Use and preparation of this compound and its salts, including the mono-lactic acid salt, are described in U.S. Pat. Nos. 6,605,617, 6,774,237, 7,335,774, and 7,470,709, and in U.S. patent application Ser. Nos. 10/982,757, 10/982,543, and 10/706,328, and in the published PCT applications WO2006/127926, published on Nov. 30, 2006 and WO2009/115562 published on Sep. 24, 2009, each of which is incorporated herein by reference in its entirety. Crystalline forms and their preparations are described in U.S.11/915005, in particular Form B.

Based on PK and safety data of COMPOUND I from Phase I and II studies, the 500 mg/day on a 5 days on/2 days off dosing schedule has been selected for Treatment Groups 1 (normal hepatic function) and 2 (mild hepatic impairment) to ensure optimal systemic exposure for the cancer patients of these two groups. Indeed due to its properties, COMPOUND I showed a time dependent plasma pharmacokinetic across all doses tested from 25 to 600 mg following continuous daily dosing regimen and the prolonged over-proportional exposure of COMPOUND I was observed at the doses of 400 mg/day and above. The 5 days on/2 days off dosing schedule was introduced to prevent such prolonged and over proportional accumulation in COMPOUND I exposure with dose escalation. For patients having moderate hepatic impairment, there is no data available as whether COMPOUND I can be administered to those patients at the same dose as the normal hepatic and mild hepatic impaired patients. Patient with hepatic impairment might be at higher risk to have a decreased ability to eliminate COMPOUND I. Decrease drug clearance as a result of impaired organ function can lead to an increased systemic exposure and possible toxicity.

The inventors of the present case are solving the current problem with the present invention. The present invention pertains to COMPOUND I for use in the treatment of cancer in a patient in need thereof wherein said patient a moderate hepatic impaired patient and wherein the dose administered is 400 mg per day and the dose schedule is 5 days on and 2 days off.

The present invention pertains to COMPOUND I for use in the treatment of cancer in a patient in need thereof wherein said patient a moderate hepatic impaired patient and wherein the dose administered is 500 mg per day and the dose schedule is 5 days on and 2 days off.

The present invention pertains to COMPOUND I for use in the treatment of cancer in a patient in need thereof wherein said patient a moderate hepatic impaired patient and wherein the dose administered is 300 mg per day and the dose schedule is 5 days on and 2 days off.

The present invention pertains to methods of treating a patient having a cancer by administering 4-amino-5-fluoro-3-[6-(4-methylpiperazin-1-yl)-1H-benzimidazol-2-yl]-1H-quinolin-2-one or a tautomer or a salt of either of them, wherein the patient is a moderate hepatic impaired patient and wherein the dose is 300 mg, 400 mg or 500 mg per day and the dose schedule is 5 days on and 2 days off.

The present invention pertains to the use of 4-amino-5-fluoro-3-[6-(4-methylpiperazin-1-yl)-1H-benzimidazol-2-yl]-1H-quinolin-2-one or a tautomer or a salt of either of them for the preparation of a medicament for the treatment of cancer wherein the patient is moderate hepatic impaired patient and the dose administered is 300 mg or 400 mg or 500 mg per day and the dose schedule is 5 days on and 2 days off.

Cancers treated according to the present invention include solid tumors, such as renal, breast, bladder, prostate cancer, and multiple myeloma.

According to the present invention, a moderate hepatic impaired patient is a patient having the following plasma characteristics : 1.5×ULN<TBL≦3.0×ULN and/or AST and ALT≦5×ULN wherein ULN means upper limit normal, TBL means total bilirubin, AST means aspartate transaminase, ALT means alanine transaminase.
Alanine transaminase (ALT), also called Serum Glutamic Pyruvate Transaminase (SGPT) or Alanine aminotransferase (ALAT) is an enzyme present in hepatocytes (liver cells). When a cell is damaged, it leaks this enzyme into the blood, where it is measured. ALT rises dramatically in acute liver damage, such as viral hepatitis or paracetamol (acetaminophen) overdose. Elevations are often measured in multiples of the upper limit of normal (ULN). The reference range for ALT is 9 to 40 IU/L (international units per liter).
Aspartate transaminase (AST) also called Serum Glutamic Oxaloacetic Transaminase (SGOT) or aspartate aminotransferase (ASAT) is similar to ALT in that it is another enzyme associated with liver parenchymal cells. It is raised in acute liver damage. The reference range for AST id 10 to 35 IU/L.
Bilirubin is a breakdown product of heme (a part of hemoglobin in red blood cells). The liver is responsible for clearing the blood of bilirubin. It does this by the following mechanism: Bilirubin is taken up into hepatocytes, conjugated (modified to make it water-soluble), and secreted into the bile, which is excreted into the intestine. Increased total bilirubin causes jaundice, and can signal a number of problems. Problems with the liver, which are reflected as deficiencies in bilirubin metabolism (e.g., reduced hepatocyte uptake, impaired conjugation of bilirubin, and reduced hepatocyte secretion of bilirubin). The normal level of total bilirubin is for example, the reference range of 0.2-1.2 mg/dL.
The person skilled in the art knows means, measurement methods and kits to measure the blood and/or plasma levels of AST, ALT and/or TBL, e.g. according to the FDA and/or the EMEA standard and knows how to stratify a patient as moderate impaired patient based on the results of the measurements of AST, ALT and/or TBL.

When COMPOUND I is administered as a salt, the dose represents milligrams of 4-amino-5 -fluoro-3-[6-(4-methylpiperazin-1-yl)-1H-benzimidazol-2-yl]-1H-quinolin-2-one. Suitable salts include any pharmaceutically acceptable salt, especially the lactate salt form, such as the mono-lactic acid salt form. Additional pharmaceutically acceptable salts are known to those of skill in the art.

According to the present invention the lactate salt of COMPOUND I can be 4-amino-5-fluoro-3-[6-(4-methylpiperazin-1-yl)-1H-benzimidazol-2-yl]-1H-quinolin-2-one lactate in crystalline Form B, as described in U.S. 11/915005.

### EXAMPLE 1

Pharmacokinetics of Compound I (4-amino-5-fluoro-3-[6-(4-methylpiperazin-1-yl)-1H-benzimidazol-2-yl]-1H-quinolin-2-one or a tautomer thereof e.g. in the lactic acid salt form thereof), in adult cancer patients with normal and moderate impaired hepatic functions

Primary objective: To evaluate the effects of mild or moderate hepatic impairment versus normal hepatic function on the pharmacokinetics of COMPOUND I in patients with advanced solid tumor

### Secondary Objectives:

To assess the safety and tolerability of COMPOUND I administration on a 5 days on/2 days off dosing schedule in adult cancer patients with mild or moderate hepatic impairment compared to patients with normal hepatic function;
To assess pharmacokinetics (PK), safety and tolerability of COMPOUND I administration on a 5 days on/2 days off dosing schedule in adult cancer patients with moderate hepatic impairment, To explore the relationship between PK and hepatic functional abnormalities (i.e. bilirubin, (alanine aminotransferease/glutamic pyruvic transaminase/GPT)/ALT/AST, and Child-Pugh classification) using regression analysis as appropriate
To evaluate the preliminary anti-tumor activity of COMPOUND I in the studied patient population
Exploratory objectives: To determine the plasma protein-binding fraction of COMPOUND I (pre-dose sample) in patients with mild, moderate or severe hepatic impairment versus normal hepatic function.
Study population: Adult patients who have advanced solid tumor (except breast cancer and lymphoma) that is either refractory to the standard therapy or has no available therapies with varying degrees of hepatic impairment according to NCI guidelines and matching cancer patients with normal hepatic function.
Number of patients: Approximately 18-48 patients

### Overview of Study Design:

This is a multi-center, open-label study to assess PK of COMPOUND I at single-dose and steady state in adult cancer patients with mild or moderate hepatic impairment relative to patients with normal hepatic function. The study also assesses PK, safety and tolerability of COMPOUND I in patients with moderate hepatic impairment, if it is considered safe to evaluate this group based on the evaluation and safety outcome of the mild and moderate hepatic impairment groups.

A single dose PK of COMPOUND I is collected from patients with normal and impaired hepatic function. Due to the time-dependent PK of COMPOUND I (auto-induction of CYP1A1/A2), steady state PK of COMPOUND I is also determined following multiple dosing of COMPOUND I for 3 weeks or 4 weeks (in case a patient missed or was unable to have a satisfying PK sampling following 3 weeks of dosing) in these patients. The study consists of 4 treatment groups, including Treatment Groups 1 (normal hepatic function) and 2 (mild hepatic impairment), 3 (moderate hepatic impairment). All treatment groups enroll patients with any solid tumor except breast cancer and lymphoma.

Treatment Group 3 (moderate hepatic dysfunction) by selecting 400 mg/day (5 days on/2 days off dose schedule) as the initial dose. However, other dose levels (500 mg/day or 300 mg/day) may be explored in patients with moderate hepatic dysfunction based on safety, tolerability, and PK data obtained during this study.
Treatment duration includes a single-dose PK period with a single dose of COMPOUND I administrated on Day 1 of Week 1 and multiple-dose treatment period with COMPOUND I, e.g. on a 5 days on/2 days off dosing regimen, starting on Day 4 of Week 1 until disease progression, e.g. assessed by RECIST 1.1, unacceptable toxicity, death or discontinuation from the study treatment for any other reason.
Approximately 18-48 patients are enrolled in 3 treatments groups based on their total bilirubin and AST/ALT levels at baseline/screening. Table below details patient allocation and treatment dose for each treatment group. The enrollment to the Treatment Groups 1-3 are in parallel, with at least 6 evaluable patients per treatment group. The mild hepatic impairment group requires 6-12 evaluable patients. The moderate hepatic impairment group may require 6-18 evaluable patients.
At least 6 patients are evaluated for PK at the identified tolerated COMPOUND I dose. To the extent possible, the enrollment to Treatment Group 1 (control) is comparable (or similar) to the enrollment to Treatment Groups 2 (mild hepatic impairment) and 3 (moderate hepatic impairment), with respect to age (±10 years) and body weight (±10 Kg). Therefore, the enrollment of Treatment Group 1 (normal hepatic function) remain open until the enrollment in the mild and moderate hepatic impairment groups is complete, and a sufficient number of matching controls has been achieved for comparison, with a minimum of 6 evaluable patients in this treatment group. In case the starting dose is not tolerated, a dose de-escalation to 400 mg is allowed for Treatment Group 2 (mild hepatic impairment), and to 300 mg for Treatment Group 3 (moderate hepatic impairment). Dose-limiting toxicities (DLT) criteria is used to guide dose escalation or dose de-escalation to identify the tolerated COMPOUND I dose in Treatment Groups 2 and 3 based on the dose levels assessed in this study. An evaluable patient is required to complete both safety (DLT evaluation) and PK assessments. A patient is considered PK evaluable if a complete PK profile is obtained at the single dose PK period and at steady state. A patient is considered evaluable for DLT if the patient completed 4 weeks of COMPOUND I treatment receiving at least 16 doses of the planned dose on a 5 days on/2 days off dosing regimen, or is discontinued from COMPOUND I treatment due to the DLT within 4 weeks on a 5 days on/2 days off dosing regimen.

| | | | Total | | | |
|---|---|---|---|---|---|---|
| | | | Bilirubin | | Dose Level 1 | Dose Level 2 |
| | | | Level | ALT/AST | (mg/day) | (mg/day) |
| | Degree of | | (TBL) | Level | (5 days on/ | (5 days on/ |
| Treatment | Hepatic | Patient | (Day 1 pre- | (Day 1 | 2 days | 2 days |
| Group | Impairment | population | dose) | pre-dose) | off) | off) |
| 1 | Normal | Any solid | TBL ≦ ULN | AST and | 500 mg | N/A |
| | hepatic | tumors* | | ALT ≦ | | |
| | function | | | ULN | | |
| | (control | | | | | |
| | group) | | | | | |
| 2 | Mild hepatic | Any solid | TBL ≦ ULN | ALT | 500 mg | 400 mg |
| | Impairment | tumors* | | and/or AST > | | |
| | | | | ULN | | |
| | | | | (with both | | |
| | | | | AST and | | |
| | | | | ALT ≦ 5 × | | |
| | | | | ULN) | | |
| | | Any solid | 1.0 × ULN < | AST and | 500 mg | 400 mg |
| | | tumors* | TBL ≦ | ALT ≦ 5 × | | |
| | | | 1.5 × ULN | ULN | | |
| 3 | Moderate | Any solid | 1.5 × ULN < | AST and | 400 mg | 300 mg |
| | hepatic | tumors* | TBL ≦ | ALT ≦ 5 × | | or |
| | impairment | | 3.0 × ULN | ULN | | 500 mg |

*any solid tumor except breast cancer and lymphoma

### Pharmacokinetic (PK) and Protein Binding Assessments:

For PK assessment: A total of 17 time points and approximately 34 mL of blood per patient is collected after starting COMPOUND I treatment. Blood samples (2 mL per sample) is taken at the following time-points:
Single-dose PK period (Day 1 of Week 1 following a single dose): predose, 2, 4, 6, 8, 24, 48, and 72 hr post dose;
Steady State (Day 1 Week 4 following multiple doses on a 3^{rd} 5 days on/2 days off dosing schedule): predose, 2, 4, 6, 8, 24, 48, and 72 hr post dose.
In case the patient missed or was not able to have a satisfying Week 4 PK sampling (e.g., due to dose interruption, patient vomiting, etc.), the steady state PK can be collected on Day 1 to Day 4 Week 5 following multiple dosing on a 4^{th} 5 days on/2 days off dosing schedule): predose, 2, 4, 6, 8, 24, 48, and 72 hr post dose.
Trough concentration (Day 1 Week 7): pre-dose

For protein binding assessment: Sample is collected at predose on Week 1 Day 1 and at predose on Week 4 Day 1 (or Week 5 Day 1, if applicable)

Safety and tolerability assessments:
Routine safety monitoring of adverse events (AEs) and serious adverse events (SAEs);
Laboratory testing of hematology (hemoglobin, WBC, platelets, and differentials), serum chemistry (urea/BUN, creatinine, total and direct bilirubin, ALT, AST, alkaline phosphatase, gamma glutamyl transpeptidase, albumin, calcium, sodium, magnesium, phosphorus, glucose, LDH, amylase, and lipase), coagulation (PT/INR, PTT and fibrinogen), and urinalysis;
Measurement of vital signs, performance status, and physical examination;
ECG evaluation (predose (triplicate with 5-10 minutes apart), and within 1 hour after 2h and 6 h postdose PK sampling);
MUGA/ECHO scan, and HBV and HCV screening in HCC patients;
Efficacy assessments is performed every 8 weeks based on the radiological tumor measurements (e.g. computed tomography or magnetic resonance imaging scan) using RECIST 1.1
Other assessments include evaluation of plasma biomarkers for FGFR and VEGFR inhibition, and identification of patients who might benefit from treatment via biomarker analyses from blood plasma samples.

### Data Analysis:

Non compartmental analysis is conducted on full PK profile of COMPOUND I. Summary statistics and mean (SD) plots is presented for COMPOUND I plasma concentration at each scheduled time point by hepatic group and dose for both single dose and steady state. Pharmacokinetic parameters such as Cmax, Tmax, AUClast, single-dose AUCinf, HL, Vz/F, CL/F is summarized by hepatic group and dose. Mild and moderate hepatic impairment groups is compared to the control group (normal hepatic function) for the PK parameters including Cmax, Tmax, AUClast, and single-dose AUCinf. A fixed effect ANOVA model is fit to the log-transformed dose-normalized primary PK parameters (Cmax, AUClast and single-dose AUCinf) from control, mild and moderate hepatic impairment groups, with hepatic function as the fixed effect if dose proportionality (DP) is a reasonable assumption. A point estimate and the corresponding 90% confidence interval for the mean difference between the control group and each hepatic impairment group is calculated. This is anti-logged to obtain the point estimate and 90% confidence interval for the ratio of the geometric means on the untransformed scale. The primary analysis is performed as described if there is further evidence for the DP assumption. Otherwise, other linear and/or non-linear model based analysis to assess the impact of hepatic function on COMPOUND I PK parameters may be employed as appropriate. The effect of baseline covariates such as age and weight is investigated, as necessary. In addition, PK data generated from this study contribute to the future meta-analysis for exploratory population PK of COMPOUND I in patients with normal or moderate hepatic function.
Safety analysis consist of AE summaries (frequency tables) and listings, laboratory abnormalities summary (shift tables for baseline to worst post-baseline) and flagging of notable values in listings, and listing of other tests (e.g., electrocardiogram or vital signs). The Safety Set is used for safety analysis. DLTs is listed using the dose-determining set.
Statistical considerations/sample size calculation: Sample size for this study is primarily driven by feasibility and simple dose finding procedure to identify the tolerated COMPOUND I dose in Treatment Groups 2 and 3 based on the dose levels assessed in this study. Approximately 18-48 patients are enrolled in study, with a minimum of 6 evaluable patients in Treatment Groups 1, 2 and 3. The inter-subject coefficient of variation (CV %) for AUC was around 50% on the 500 mg/day on a 5 days on/2 days off dosing schedule. Using an inter-subject CV % estimate of 50% and a sample size of 6 per treatment group, the precision or half-width of 90% confidence intervals (CI) for (impaired hepatic function)−(normal hepatic function) comparison on the log scale extends 0.494 from the observed difference in means. This calculation is based on a two sample t-test with a type I error rate of 10%. No adjustments were made for multiple comparisons. The above half width translates into the following 90% confidence intervals (CI) of pharmacokinetic parameter ratios assuming different observed ratios.

| | Lower limit of 90% | Upper limit of 90% |
|---|---|---|
| Ratio | CI | CI |
| 0.5 | 0.31 | 0.82 |
| 1.00 | 0.61 | 1.64 |
| 1.50 | 0.92 | 2.46 |
| 2.00 | 1.22 | 3.28 |

While certain embodiments have been illustrated and described, it should be understood that changes and modifications can be made therein in accordance with ordinary skill in the art without departing from the present technology in its broader aspects as defined in the following claims.

1. Method of treating a cancer in patient in need thereof by administering 4-amino-5-fluoro-3-[6-(4-methylpiperazin-1-yl)-1H-benzimidazol-2-yl]-1H-quinolin-2-one, or a tautomer thereof, or a salt of any one of them, wherein the patient is a moderate hepatic impaired patient.

2. Method according to claim 1 wherein -amino-5-fluoro-3-[6-(4-methylpiperazin-1-yl)-1H-benzimidazol-2-yl]-1H-quinolin-2-one, or a tautomer thereof, or a salt of any one of them is administered at the dose, is 300 mg, 400mg or 500 mg per day and the dose schedule is 5 days on and 2 days off.

3. Method of claim 1 wherein the moderate hepatic impaired patient is characterized by the following plasma characteristics: 1.5×ULN<TBL 3.0×ULN and/or AST and ALT 5×ULN wherein ULN means upper limit normal, TBL means total bilirubin, AST means aspartate transaminase, ALT means alanine transaminase.

4. Method according to claim 1 wherein 4-amino-5-fluoro-3-[6-(4-methylpiperazin-1-yl)-1H-benzimidazol-2-yl]-1H-quinolin-2-one or a tautomer thereof, is in e lactate salt form thereof.

5. Method of claim 4 wherein the lactic acid is D,L-lactic acid.

6. Method according to claim 1 wherein 4-amino-5-fluoro-3-[6-(4-methylpiperazin-1-yl)-1H-benzimidazol-2-yl]-1H-quinolin-2-one lactic acid in in crystalline Form B.
